(19) United States
(12) Patent Application Publication
(10) Pub. No.: US 2026/0161071 A1
(43) Pub. Date: Jun. 11, 2026

(54) SEMICONDUCTOR PHOTORESIST COMPOSITION AND METHOD OF FORMING PATTERNS USING THE COMPOSITION

(71) Applicant: SAMSUNG SDI CO., LTD., Yongin-si (KR)

(72) Inventors: Jimin KIM, Suwon-si (KR); Seung-Wook SHIN, Suwon-si (KR); Minyoung LEE, Suwon-si (KR); Yaeun SEO, Suwon-si (KR); Eunmi KANG, Suwon-si (KR); Sumin JANG, Suwon-si (KR); Kyoungah OH, Suwon-si (KR); Jihyun YOON, Suwon-si (KR); Yunju CHAE, Suwon-si (KR); Seongyeon HWANG, Suwon-si (KR); Hujeong YOON, Suwon-si (KR)

(21) Appl. No.: 19/179,632

(22) Filed: Apr. 15, 2025

(30) Foreign Application Priority Data
Jul. 31, 2024 (KR) 10-2024-0102081

(51) Int. Cl. G03F 7/004; C07C 53/122; C07C 55/12; C07C 61/08; C07C 63/04; C07F 7/22; H10P 50/00; H10P 76/20

(52) U.S. Cl. CPC G03F 7/0042; C07C 53/122; C07C 55/12; C07C 61/08; C07C 63/04; C07F 7/2224; H10P 76/204; H10P 50/71; H10P 50/73

(57) ABSTRACT

A semiconductor photoresist composition includes an organometallic compound; a linear carboxylic acid compound including at least one carboxyl group; a cyclic carboxylic acid compound represented by Chemical Formula 1; and a solvent. The details of Chemical Formula 1 are as described in the specification. A method of forming patterns utilizes the semiconductor photoresist composition.

### CROSS-REFERENCE TO RELATED APPLICATION

The present application claims priority to and the benefit of Korean Patent Application No. 10-2024-0102081, filed on Jul. 31, 2024, in the Korean Intellectual Property Office, the entire content of which is incorporated herein by reference.

### BACKGROUND

### 1 Field

This disclosure relates to a semiconductor photoresist composition and a method of forming patterns using the same.

### 2. Description of the Related Art

Extreme ultraviolet (EUV) lithography has gained attention as an important (e.g., essential) technology for manufacturing a next generation semiconductor device. EUV lithography is a pattern-forming technology that uses an EUV ray with a wavelength of 13.5 nm as an exposure light source. In EUV lithography, extremely fine patterns (e.g., less than or equal to 20 nm) may be formed through an exposure process during the manufacturing of semiconductor devices.

The realization of extreme ultraviolet (EUV) lithography depends on development of compatible photoresists that can achieve a spatial resolution of less than or equal to 16 nm. Currently, efforts are underway to address the limitations of related art chemically amplified (CA) photoresists, such as in resolution, photospeed, and/or feature roughness (also referred to as a line edge roughness or LER)), to meet the specifications for the next generation device.

Intrinsic image blurring due to an acid catalyzed reaction in these polymer-type (kind) photoresists limits a resolution in small feature sizes, a phenomenon known in electron beam (e-beam) lithography. The chemically amplified (CA) photoresists are designed for high sensitivity, but their typical elemental makeups reduce light absorbance of the photoresists at a wavelength of 13.5 nm and thus decrease their sensitivity under EUV exposure.

In addition, the CA photoresists may have difficulties with small feature sizes due to roughness issues. Experimental results show that line edge roughness (LER) increases as photospeed decreases partially due to inherent characteristics of acid catalyst processes. Accordingly, a novel high-performance photoresist is desired or required in the semiconductor industry to address these defects and problems of the CA photoresists.

In order to overcome the aforementioned drawbacks of the chemically amplified (CA) organic photosensitive composition, research has been conducted on inorganic photosensitive compositions. These inorganic photosensitive compositions are mainly used for negative tone patterning and have resistance against removal by a developer composition due to chemical modification through a nonchemical amplification mechanism. Inorganic photosensitive compositions contain inorganic elements with higher EUV absorption rates than hydrocarbons, and thus may secure or ensure suitable sensitivity through the nonchemical amplification mechanism.

Additionally, inorganic photosensitive compositions are less sensitive to stochastic effects and may have low line edge roughness and fewer defects.

Inorganic photoresists based on peroxopolyacids of tungsten mixed with tungsten, niobium, titanium, and/or tantalum have been reported as radiation sensitive materials for patterning (see also, U.S. Pat. No. 5,061,599; and H. Okamoto, T. Iwayanagi, K. Mochiji, H. Umezaki, T. Kudo, Applied Physics Letters, 49(5), 298-300, 1986, the entire content of each of which is incorporated herein by reference).

These materials are effective for patterning large pitches for bilayer configuration as far ultraviolet (deep UV), X-ray, and electron beam sources. More recently, cationic hafnium metal oxide sulfate (HfSOx) materials along with a peroxo complexing agent has been used to image a 15 nm half-pitch (HP) through projection EUV exposure, and impressive performance has been obtained (see also, US 2011-0045406; and J. K. Stowers, A. Telecky, M. Kocsis, B. L. Clark, D. A. Keszler, A. Grenville, C. N. Anderson, P. P. Naulleau, Proc. SPIE, 7969, 796915, 2011, the entire content of each of which is incorporated herein by reference). This system exhibits better performance as a non-CA photoresist and has a practicable photospeed close to the requirement for an EUV photoresist. However, the hafnium metal oxide sulfate material having the peroxo complexing agent has a few practical drawbacks. First, these materials are coated in a mixture of corrosive sulfuric acid/hydrogen peroxide and may have insufficient shelf-life stability. Second, as a composite mixture, a structural change thereof for performance improvement is not easy. Third, development has to be performed in a tetramethylammonium hydroxide (TMAH) solution at a height (e.g., extremely high) concentration of 25 wt % and/or the like.

Recently, active research has been conducted on molecules (e.g., materials) including tin, which have excellent or suitable absorption of extreme ultraviolet rays. Among these, organotin polymers dissociate alkyl ligands by light absorption or secondary electrons produced thereby, and crosslink with adjacent chains through oxo bonds, enabling negative tone patterning that resists removal by an organic developer. This organotin polymer exhibits greatly improved sensitivity as well as maintaining suitable resolution and line edge roughness, but further improvement in patterning characteristics is needed or desired for commercial availability.

### SUMMARY

An aspect according to some embodiments is directed toward a semiconductor photoresist composition having (with) improved coatability and storage stability while maintaining excellent or suitable sensitivity characteristics.

An aspect according to some embodiments is directed toward a method of forming patterns using the semiconductor photoresist composition.

Additional aspects will be set forth in part in the description which follows and, in part, will be apparent from the description, or may be learned by practice of the presented embodiments of the disclosure.

A semiconductor photoresist composition according to some embodiments includes an organometallic compound, a linear carboxylic acid compound including at least one carboxyl group, a cyclic carboxylic acid compound represented by Chemical Formula 1, and a solvent.

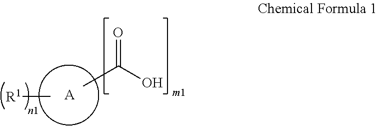

In Chemical Formula 1,
R¹ may be a substituted or unsubstituted C1 to C20 alkyl group, a substituted or unsubstituted C2 to C20 alkenyl group, a substituted or unsubstituted C2 to C20 alkynyl group, a substituted or unsubstituted C3 to C20 cycloalkyl group, a substituted or unsubstituted C3 to C20 cycloalkenyl group, a substituted or unsubstituted C6 to C30 aryl group, or a substituted or unsubstituted C7 to C30 arylalkyl group,
n1 may be the number of substituents from 1 to the maximum number substituted for A, that is, n1 may be an integer selected from among 1 to a maximum number of substituents allowed for A,
ring A may be a substituted or unsubstituted C3 to C20 non-aromatic carbocyclic group or a substituted or unsubstituted C6 to C30 aromatic carbocyclic group, and
m1 may be an integer selected from among 1 to 3.

A method of forming patterns according to some embodiments includes forming an etching-objective layer on a substrate, coating the semiconductor photoresist composition on the etching-objective layer to form a photoresist layer, patterning the photoresist layer to form a photoresist pattern and etching the etching-objective layer using the photoresist pattern as an etching mask.

The semiconductor photoresist composition according to some embodiments can improve surface roughness by enhancing coating properties and provide a photoresist pattern with improved solution stability (e.g., stability in a solution), thereby maintaining the same performance of sensitivity, LER, and pattern line width for a certain period of time.

### BRIEF DESCRIPTION OF THE DRAWINGS

The accompanying drawings are included to provide a further understanding of the present disclosure and are incorporated in and constitute a part of this specification. The drawings illustrate embodiments of the present disclosure and, together with the description, serve to explain principles of the present disclosure. In the drawings:

FIGS. 1A-1E are cross-sectional views for explaining a method of forming patterns using a semiconductor photoresist composition according to some example embodiments.

### DETAILED DESCRIPTION

Hereinafter, referring to the drawings, embodiments are described in more detail. In the following description of the present disclosure, the functions or constructions known in the related art will not be described in order to clarify the present disclosure.

Throughout the disclosure, the same or similar configuration elements are designated by the same reference numerals. Also, because the size and thickness of each configuration shown in the drawing are arbitrarily shown for better understanding and ease of description, the present disclosure is not necessarily limited thereto.

In the drawings, the thickness of layers, films, panels, regions, and/or the like, may be enlarged for clarity. In the drawings, the thickness of a part of layers or regions, and/or the like, may be exaggerated for clarity. It will be understood that if (e.g., when) an element such as a layer, film, region, or substrate is referred to as being “on” another element, it can be directly on the other element or intervening elements may also be present.

As used herein, the term “substituted” refers to replacement of a hydrogen atom by deuterium, a halogen, a hydroxyl group, a carboxyl group, a thiol group, a cyano group, a nitro group, —NRR′ (wherein, R and R′ may each independently be hydrogen, a substituted or unsubstituted C1 to C30 saturated or unsaturated aliphatic hydrocarbon group, a substituted or unsubstituted C3 to C30 saturated or unsaturated alicyclic hydrocarbon group, or a substituted or unsubstituted C6 to C30 aromatic hydrocarbon group), —SiRR′R″ (wherein, R, R′, and R″ may each independently be hydrogen, a substituted or unsubstituted C1 to C30 saturated or unsaturated aliphatic hydrocarbon group, a substituted or unsubstituted C3 to C30 saturated or unsaturated alicyclic hydrocarbon group, or a substituted or unsubstituted C6 to C30 aromatic hydrocarbon group), a C1 to C30 alkyl group, a C1 to C10 haloalkyl group, a C1 to C10 alkylsilyl group, a C3 to C30 cycloalkyl group, a C6 to C30 aryl group, a C1 to C20 alkoxy group, a C1 to C20 sulfide group, and/or a (e.g., any suitable) combination thereof. the term “unsubstituted” refers to non-replacement of a hydrogen atom by another substituent and remaining as the hydrogen atom.

As used herein, if (e.g., when) a definition is not otherwise provided, the term “alkyl group” refers to a linear or branched aliphatic hydrocarbon group. The alkyl group may be “saturated alkyl group” without any double bond or triple bond.

The alkyl group may be a C1 to C8 alkyl group. For example, the alkyl group may be a C1 to C7 alkyl group, a C1 to C6 alkyl group, or a C1 to C5 alkyl group. For example, the C1 to C5 alkyl group may be a methyl group, an ethyl group, a propyl group, an isopropyl group, an n-butyl group, an isobutyl group, a sec-butyl group, a tert-butyl group, or a 2,2-dimethylpropyl group.

As used herein, if (e.g., when) a definition is not otherwise provided, the term “cycloalkyl group” refers to a monovalent cyclic aliphatic hydrocarbon group.

The cycloalkyl group may be a C3 to C8 cycloalkyl group, for example, a C3 to C7 cycloalkyl group, a C3 to C6 cycloalkyl group, a C3 to C5 cycloalkyl group, or a C3 to C4 cycloalkyl group. For example, the cycloalkyl group may be a cyclopropyl group, a cyclobutyl group, a cyclopentyl group, or a cyclohexyl group, but the present disclosure is not limited thereto.

As used herein, the term “aryl group” refers to a cyclic substituent in which all atoms in the cyclic substituent have a p-orbital and these p-orbitals are conjugated and may include a monocyclic or fused ring polycyclic (i.e., rings sharing adjacent pairs of carbon atoms) functional group.

As used herein, the term “heteroaryl group” may refer to an aryl group including at least one heteroatom selected from among N, O, S, P, and Si. Two or more heteroaryl groups may be linked by a sigma bond directly, or if (e.g., when) the heteroaryl group includes two or more rings, the two or more rings may be fused. If (e.g., when) the heteroaryl group is a fused ring, each ring may include one to three heteroatoms.

As used herein, unless otherwise defined, the term “alkenyl group” refers to an aliphatic unsaturated alkenyl group including at least one double bond as a linear or branched aliphatic hydrocarbon group.

As used herein, unless otherwise defined, the term “alkynyl group” refers to an aliphatic unsaturated alkynyl group including at least one triple bond as a linear or branched aliphatic hydrocarbon group.

A semiconductor photoresist composition according to some example embodiments is described.

The semiconductor photoresist composition according to some example embodiments may include an organometallic compound, a linear carboxylic acid compound, a cyclic carboxylic acid compound represented by Chemical Formula 1, and a solvent.

The semiconductor photoresist composition according to the present disclosure is a composition including an organometallic compound, two types (kinds) of carboxylic acid compounds, and a solvent. By including the linear carboxylic acid compound and substituted cyclic carboxylic acid compound, the sensitivity to extreme ultraviolet rays in a pattern formed using the composition may be increased and the pattern roughness may be improved.

The cyclic carboxylic acid compound may be represented by Chemical Formula 1.

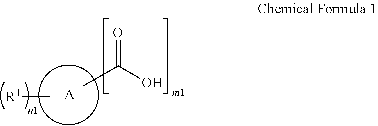

In Chemical Formula 1,
R¹ may be a substituted or unsubstituted C1 to C20 alkyl group, a substituted or unsubstituted C2 to C20 alkenyl group, a substituted or unsubstituted C2 to C20 alkynyl group, a substituted or unsubstituted C3 to C20 cycloalkyl group, a substituted or unsubstituted C3 to C20 cycloalkenyl group, a substituted or unsubstituted C6 to C30 aryl group, or a substituted or unsubstituted C7 to C30 arylalkyl group,
n1 may be the number of substituents (R¹) from 1 to the maximum number of substituents (R¹) for A, that is, n1 may be an integer selected from among 1 to a maximum number of R¹ allowed for A,
ring A may be a substituted or unsubstituted C3 to C20 non-aromatic carbocyclic group or a substituted or unsubstituted C6 to C30 aromatic carbocyclic group, and
m1 may be an integer selected from among 1 to 3.

In the present specification, a C3 to C20 non-aromatic carbocyclic group refers to a saturated or unsaturated cyclic group having 3 to 20 carbons as ring forming atoms. The C3 to C20 non-aromatic carbocyclic group may be a monocyclic group or a polycyclic group.

In the present specification, the C6 to C30 aromatic carbocyclic group refers to an aromatic ring having 6 to 20 carbons as ring forming atoms. For example, the C6 to C30 aromatic carbocyclic group may be selected from among a benzene group, a naphthalene group, an anthracene group, a phenanthrene group, a triphenylene group, a pyrene group, and a chrysene group, but the present disclosure is not limited thereto.

The non-aromatic carbocyclic group and aromatic carbocyclic group may be modified in one or more suitable ways, such as being a divalent group, a trivalent group, or a tetravalent group, depending on the number of linked substituents.

In some embodiments, the ring A may be a substituted or unsubstituted cyclopentane group, a substituted or unsubstituted cyclohexane group, a substituted or unsubstituted heptane group, a substituted or unsubstituted octane group, a substituted or unsubstituted cyclopentene group, a substituted or unsubstituted cyclohexene group, a substituted or unsubstituted benzene group, a substituted or unsubstituted naphthalene group, a substituted or unsubstituted anthracene group, a substituted or unsubstituted phenanthrene group, a substituted or unsubstituted pyrene group, a substituted or unsubstituted triphenylene group, and/or a (e.g., any suitable) combination thereof.

In some embodiments, the cyclic carboxylic acid compound may be represented by Chemical Formula 1A or Chemical Formula 1B.

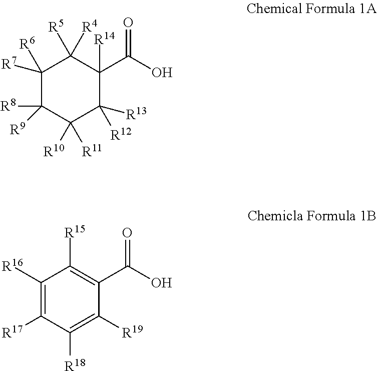

In Chemical Formula 1A and Chemical Formula 1B,
R⁴ to R¹⁹ may each independently be hydrogen, a substituted or unsubstituted C1 to C20 alkyl group, a substituted or unsubstituted C2 to C20 alkenyl group, a substituted or unsubstituted C2 to C20 alkynyl group, a substituted or unsubstituted C3 to C20 cycloalkyl group, a substituted or unsubstituted C3 to C20 cycloalkenyl group, a substituted or unsubstituted C6 to C30 aryl group, or a substituted or unsubstituted C7 to C30 arylalkyl group,
at least one of (e.g., at least one selected from among) R⁴ to R¹⁴ may be a substituted or unsubstituted C1 to C20 alkyl group, a substituted or unsubstituted C2 to C20 alkenyl group, a substituted or unsubstituted C2 to C20 alkynyl group, a substituted or unsubstituted C3 to C20 cycloalkyl group, a substituted or unsubstituted C3 to C20 cycloalkenyl group, a substituted or unsubstituted C6 to C30 aryl group, or a substituted or unsubstituted C7 to C30 arylalkyl group, and
at least one of R¹⁵ to R¹⁹ may be a substituted or unsubstituted C1 to C20 alkyl group, a substituted or unsubstituted C2 to C20 alkenyl group, a substituted or unsubstituted C2 to C20 alkynyl group, a substituted or unsubstituted C3 to C20 cycloalkyl group, a substituted or unsubstituted C3 to C20 cycloalkenyl group, a substituted or unsubstituted C6 to C30 aryl group, or a substituted or unsubstituted C7 to C30 arylalkyl group.

In some embodiments, at least one of R⁴ to R¹⁴ may be a substituted or unsubstituted methyl group, a substituted or unsubstituted ethyl group, a substituted or unsubstituted propyl group, a substituted or unsubstituted iso-propyl group, a substituted or unsubstituted butyl group, a substituted or unsubstituted sec-butyl group, a substituted or unsubstituted iso-butyl group, a substituted or unsubstituted tert-butyl group, a substituted or unsubstituted pentyl group, and/or a (e.g., any suitable) combination thereof, and

at least one of R¹⁵ to R¹⁹ may be a substituted or unsubstituted methyl group, a substituted or unsubstituted ethyl group, a substituted or unsubstituted propyl group, a substituted or unsubstituted iso-propyl group, a substituted or unsubstituted butyl group, a substituted or unsubstituted sec-butyl group, a substituted or unsubstituted iso-butyl group, a substituted or unsubstituted tert-butyl group, a substituted or unsubstituted pentyl group, and/or a (e.g., any suitable) combination thereof.

In an embodiment, the cyclic carboxylic acid compound may be any one selected from among the compounds listed in Group 1.

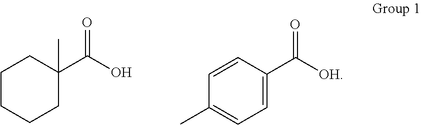

In some embodiments, the linear carboxylic acid compound may be represented by Chemical Formula 2.

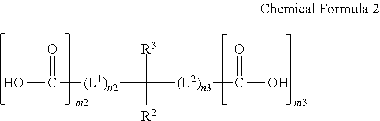

In Chemical Formula 2,
R² and R³ may each independently be hydrogen, a hydroxyl group, a carboxyl group, a substituted or unsubstituted C1 to C20 alkyl group, a substituted or unsubstituted C2 to C20 alkenyl group, a substituted or unsubstituted C2 to C20 alkynyl group, a substituted or unsubstituted C3 to C20 cycloalkyl group, a substituted or unsubstituted C3 to C20 cycloalkenyl group, a substituted or unsubstituted C6 to C30 aryl group, or a substituted or unsubstituted C7 to C30 arylalkyl group,
L¹ and L² may each independently be a single bond, a substituted or unsubstituted C1 to C10 alkylene group, a substituted or unsubstituted C2 to C20 alkenylene group, a substituted or unsubstituted C2 to C20 alkynylene group, a substituted or unsubstituted C3 to C20 cycloalkylene group, a substituted or unsubstituted C3 to C20 cycloalkenylene group, or a substituted or unsubstituted C6 to C20 arylene group,
n2 and n3 may each independently be an integer selected from among 0 to 5,
m2 and m3 may each independently be 0 or 1,
m2+m3 is an integer of greater than or equal to 1, and
if (e.g., when) n2 and n3 are each an integer greater than or equal to 2, each of L¹ and L² may be the same as or different from each other.

If (e.g., when) n2 is an integer greater than or equal to 2, each L¹ is the same as or different from each other.

If (e.g., when) n3 is an integer greater than or equal to 2, each L² is the same as or different from each other.

If (e.g., when) m2 or m3 is 0, L¹ or L² may be a monovalent substituent within substantially the same definition as R² and R³, respectively. That is, if (e.g., when) m2 or m3 is 0, the respective L¹ or L² may be hydrogen, a hydroxyl group, a carboxyl group, a substituted or unsubstituted C1 to C20 alkyl group, a substituted or unsubstituted C2 to C20 alkenyl group, a substituted or unsubstituted C2 to C20 alkynyl group, a substituted or unsubstituted C3 to C20 cycloalkyl group, a substituted or unsubstituted C3 to C20 cycloalkenyl group, a substituted or unsubstituted C6 to C30 aryl group, or a substituted or unsubstituted C7 to C30 arylalkyl group.

In some embodiments, R² and R³ may each independently be hydrogen, a hydroxyl group, a substituted or unsubstituted C1 to C10 alkyl group, a substituted or unsubstituted C2 to C10 alkenyl group, a substituted or unsubstituted C2 to C10 alkynyl group, a substituted or unsubstituted C3 to C10 cycloalkyl group, a substituted or unsubstituted C3 to C10 cycloalkenyl group, a substituted or unsubstituted C6 to C20 aryl group, or a substituted or unsubstituted C7 to C20 arylalkyl group.

In some embodiments, R² and R³ may each independently be hydrogen, a substituted or unsubstituted methyl group, a substituted or unsubstituted ethyl group, a substituted or unsubstituted propyl group, a substituted or unsubstituted iso-propyl group, a substituted or unsubstituted butyl group, a substituted or unsubstituted sec-butyl group, a substituted or unsubstituted iso-butyl group, a substituted or unsubstituted tert-butyl group, a substituted or unsubstituted pentyl group, and/or a (e.g., any suitable) combination thereof.

In some embodiments, L¹ and L² may each independently be a substituted or unsubstituted C1 to C7 alkylene group, a substituted or unsubstituted C2 to C10 alkenylene group, a substituted or unsubstituted C2 to C10 alkynylene group, a substituted or unsubstituted C3 to C10 cycloalkylene group, a substituted or unsubstituted C3 to C10 cycloalkenylene group, or a substituted or unsubstituted C6 to C12 arylene group.

In some embodiments, each of L¹ and L² may each independently be a substituted or unsubstituted methylene group, a substituted or unsubstituted ethylene group, a substituted or unsubstituted propylene group, a substituted or unsubstituted butylene group, a substituted or unsubstituted pentylene group, a substituted or unsubstituted cyclopentylene group, a substituted or unsubstituted cyclopentenylene group, a substituted or unsubstituted cyclopentadienylene group, or a substituted or unsubstituted cyclohexylene group.

In an embodiment, the linear carboxylic acid compound may be one (e.g., any one) selected from among the compounds listed in Group 2.

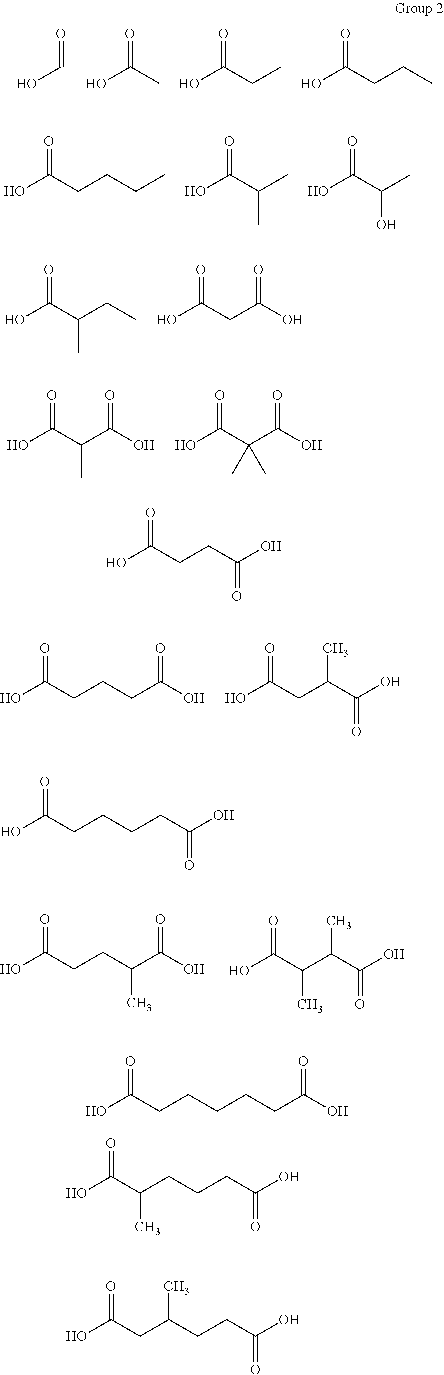

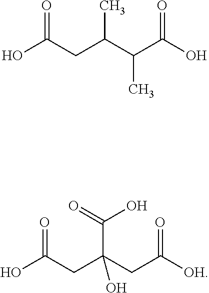

A weight ratio between the linear carboxylic acid compound and the cyclic carboxylic acid compound (linear carboxylic acid compound:cyclic carboxylic acid compound) in the semiconductor photoresist composition may be about 9:1 to about 1:9.

For example, the linear carboxylic acid compound and the cyclic carboxylic acid compound may be included in a weight ratio of about 9:1 to about 2:8, about 9:1 to about 3:7, about 8:2 to about 3:7, or about 8:2 to about 5:5. That is, the weight ratio between the linear carboxylic acid compound and the cyclic carboxylic acid compound in the semiconductor photoresist composition may be about 9:1 to about 2:8, about 9:1 to about 3:7, about 8:2 to about 3:7, or about 8:2 to about 5:5.

In an embodiment, the linear carboxylic acid compound: the cyclic carboxylic acid compound may be included in a weight ratio of about 3:1 to about 1:3 or a weight ratio of about 3:1 to about 1:1. That is, the weight ratio between the linear carboxylic acid compound and the cyclic carboxylic acid compound in the semiconductor photoresist composition may be about 3:1 to about 1:3 or a weight ratio of about 3:1 to about 1:1. The linear carboxylic acid compound and the cyclic carboxylic acid compound may be included in an amount of about 0.01 to 20 wt % based on 100 wt % of the semiconductor photoresist composition.

For example, the linear carboxylic acid compound and the cyclic carboxylic acid compound may be included in an amount of about 0.01 to about 10 wt %, about 0.02 to about 10 wt %, about 0.03 to about 10 wt %, or about 0.05 to about 10 wt % based on 100 wt % of the semiconductor photoresist composition.

The organometallic compound may be included in an amount of about 0.5 wt % to about 30 wt % based on 100 wt % of the semiconductor photoresist composition.

A semiconductor photoresist composition according to some embodiments may improve the sensitivity of the photoresist by including both the linear carboxylic acid compound and the cyclic carboxylic acid compound, and in the above content (e.g., amount) ranges.

The organometallic compound may be an organotin compound including at least one of an organooxy group and an organocarbonyloxy group.

In some embodiments, the organometallic compound may be represented by Chemical Formula 3.

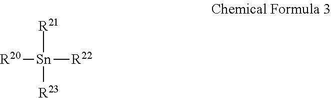

In Chemical Formula 3,
R²⁰ may be selected from among a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and a substituted or unsubstituted C7 to C30 arylalkyl group,
R²¹ to R²³ may each independently be a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, a substituted or unsubstituted C7 to C30 arylalkyl group, an alkoxy or aryloxy group (—OR^{b}, wherein R^{b} may be a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof), a carboxyl group (—O(C═O)R^{c}, wherein R^{c} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof), an alkylamido or dialkylamido group (—NR^{d}R^{e}, wherein R^{d} and R^{e} may each independently be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof), an amidato group (—NR^{f}(COR^{g}), wherein R^{f} and R^{g} may each independently be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof), an amidinato group (—NR^{h}C(NRⁱ)R^{j}, wherein R^{h}, Rⁱ, and R^{j} may each independently be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof), an alkylthio or arylthiol group (—SR^{k}, wherein R^{k} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof), or a thiocarboxyl group (—S(CO)R^{l}, wherein R^{l} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof), and
at least one selected from among R²¹ to R²³ may be selected from among an alkoxy or aryloxy group (—OR^{b}, wherein R^{b} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof), a carboxyl group (—O(C═O)R^{c}, wherein R^{c} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof), an alkylamido or dialkylamido group (—NR^{d}R^{e}, wherein R^{d} and R^{e} may each independently be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof), an amidato group (—NR^{f}(COR^{g}), wherein R^{f} and R^{g} may each independently be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof), an amidinato group (—NR^{h}C(NRⁱ)R^{j}, wherein R^{h}, Rⁱ, and R^{j} may each independently be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof), an alkylthio or arylthiol group (—SR^{k}, wherein R^{k} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof), and a thiocarboxyl group (—S(CO)R^{l}, wherein R^{l} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof).

In some embodiments, at least one selected from among R²¹ to R²³ may be selected from among an alkoxy or aryloxy group (—OR^{b}, wherein R^{b} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof), and a carboxyl group (—O(C═O)R^{c}, wherein R^{c} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof).

In some embodiments, because the compound represented by Chemical Formula 3 includes —OR^{b} or —OC(═O)R^{c} as a ligand, a pattern formed using a semiconductor photoresist composition including the compound can exhibit excellent or suitable resolution.

Additionally, the ligand of —OR^{b} or —OC(═O)R^{c} can determine the solubility of the compound represented by Chemical Formula 3 in a solvent.

In some embodiments, R²⁰ may be a substituted or unsubstituted C1 to C8 alkyl group, a substituted or unsubstituted C3 to C8 cycloalkyl group, a substituted or unsubstituted C2 to C8 aliphatic unsaturated organic group including one or more double bonds or triple bonds, a substituted or unsubstituted C6 to C20 aryl group, a substituted or unsubstituted C4 to C20 heteroaryl group, a carbonyl group, an ethoxy group, a propoxy group, and/or a (e.g., any suitable) combination thereof,
R^{b} may be a substituted or unsubstituted C1 to C8 alkyl group, a substituted or unsubstituted C3 to C8 cycloalkyl group, a substituted or unsubstituted C2 to C8 alkenyl group, a substituted or unsubstituted C2 to C8 alkynyl group, a substituted or unsubstituted C6 to C20 aryl group, and/or a (e.g., any suitable) combination thereof, and
R^{c} may be hydrogen, a substituted or unsubstituted C1 to C8 alkyl group, a substituted or unsubstituted C3 to C8 cycloalkyl group, a substituted or unsubstituted C2 to C8 alkenyl group, a substituted or unsubstituted C2 to C8 alkynyl group, a substituted or unsubstituted C6 to C20 aryl group, and/or a (e.g., any suitable) combination thereof.

In some embodiments, R²⁰ may be a methyl group, an ethyl group, a propyl group, a butyl group, an isopropyl group, a tert-butyl group, a 2,2-dimethylpropyl group, a cyclopropyl group, a cyclobutyl group, a cyclopentyl group, a cyclohexyl group, an ethenyl group, a propenyl group, a butenyl group, an ethynyl group, a propynyl group, a butynyl group, a phenyl group, a tolyl group, a xylene group, a benzyl group, a formyl group, an acetyl group, a propanoyl group, a butanoyl group, a pentanoyl group, an ethoxy group, a propoxy group, and/or a (e.g., any suitable) combination thereof,
R^{b} may be an ethyl group, a propyl group, a butyl group, an isopropyl group, a tert-butyl group, a 2,2-dimethylpropyl group, a cyclopropyl group, a cyclobutyl group, a cyclopentyl group, a cyclohexyl group, an ethenyl group, a propenyl group, a butenyl group, an ethynyl group, a propynyl group, a butynyl group, a phenyl group, a tolyl group, a xylene group, a benzyl group, and/or a (e.g., any suitable) combination thereof, and
R^{c} may be hydrogen, an ethyl group, a propyl group, a butyl group, an isopropyl group, a tert-butyl group, a 2,2-dimethylpropyl group, a cyclopropyl group, a cyclobutyl group, a cyclopentyl group, a cyclohexyl group, an ethenyl group, a propenyl group, a butenyl group, an ethynyl group, a propynyl group, a butynyl group, a phenyl group, a tolyl group, a xylene group, a benzyl group, and/or a (e.g., any suitable) combination thereof.

In some embodiments, the organometallic compound may be represented by Chemical Formula 4 or Chemical Formula 5.

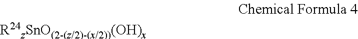

In Chemical Formula 4,
R²⁴ may be a C1 to C31 hydrocarbyl group, wherein 0<z≤2 and 0<(z+x)≤4;

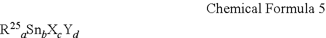

wherein, in Chemical Formula 5,
R²⁵ may be a substituted or unsubstituted C1 to C20 alkyl group, a substituted or unsubstituted C3 to C20 cycloalkyl group, a substituted or unsubstituted C2 to C20 aliphatic unsaturated organic group including one or more double bonds or triple bonds, a substituted or unsubstituted C6 to C30 aryl group, a substituted or unsubstituted C4 to C30 heteroaryl group, a carbonyl group, an ethylene oxide group, a propylene oxide group, and/or a (e.g., any suitable) combination thereof,
X may be sulfur (S), selenium (Se), or tellurium (Te),
Y may be —OR^{m} or —OC(═O)Rⁿ,
R^{m} may be a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof,
Rⁿ may be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and/or a (e.g., any suitable) combination thereof, and

a, b, c, and d may each independently be an integer from 1 to 20.

The solvent included in the semiconductor photoresist composition according to some embodiments may be an organic solvent. For example, the solvent may be aromatic compounds (e.g., xylene, toluene, and/or the like), alcohols (e.g., 4-methyl-2-pentanol, 4-methyl-2-propanol, 1-butanol, methanol, isopropyl alcohol, and/or 1-propanol), ethers (e.g., anisole, tetrahydrofuran), esters (n-butyl acetate, propylene glycol monomethyl ether acetate, ethyl acetate, and/or ethyl lactate), ketones (e.g., methyl ethyl ketone, and/or 2-heptanone), and/or a (e.g., any suitable) mixture thereof, but the present disclosure is not limited thereto.

The semiconductor photoresist composition according to some embodiments may further include a resin in addition to the aforementioned organometallic compound, linear carboxylic acid compound including at least one carboxyl group, cyclic carboxylic acid compound, and solvent.

The resin may be a phenolic resin including at least one aromatic moiety listed (e.g., including at least one aromatic moiety selected among the aromatic moieties) in Group 3.

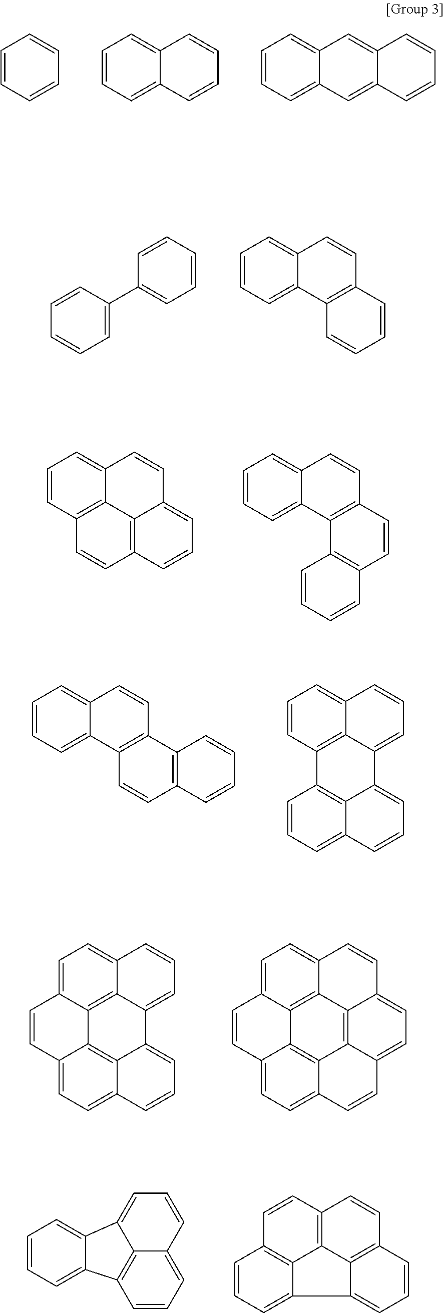

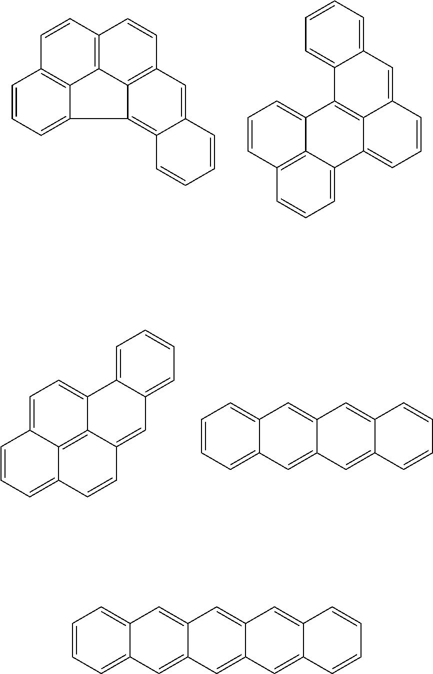

The resin may have a weight average molecular weight of about 500 to about 20,000.

The resin may be included in an amount of about 0.1 wt % to about 50 wt % based on a total 100 wt % of the semiconductor photoresist composition.

If (e.g., when) the resin is included within the above content (e.g., amount) range, the semiconductor photoresist composition can have excellent or suitable etching resistance and heat resistance.

In one or more embodiments, the semiconductor photoresist composition may be composed of the aforementioned organometallic compound, linear carboxylic acid compound including at least one carboxyl group, cyclic carboxylic acid compound, solvent, and resin.

In some embodiments, the semiconductor photoresist composition may further include one or more additives. Examples of the additives may include a surfactant, a crosslinking agent, a leveling agent, an organic acid, a quencher, and/or a (e.g., any suitable) combination thereof.

The surfactant may include for example an alkyl benzene sulfonate salt, an alkyl pyridinium salt, polyethylene glycol, a quaternary ammonium salt, and/or a (e.g., any suitable) combination thereof, but the present disclosure is not limited thereto.

The crosslinking agent may be for example a melamine-based crosslinking agent, a substituted urea-based crosslinking agent, an acryl-based crosslinking agent, an epoxy-based crosslinking agent, or a polymer-based crosslinking agent, but the present disclosure is not limited thereto. The crosslinking agent may have at least two crosslinking forming substituents, for example, the crosslinking agent may be a compound such as methoxymethylated glycoluril, butoxymethylated glycoluril, methoxymethylated melamine, butoxymethylated melamine, methoxymethylated benzoguanamine, butoxymethylated benzoguanamine, 4-hydroxybutyl acrylate, acrylic acid, urethane acrylate, acryl methacrylate, 1,4-butanediol diglycidyl ether, glycidol, diglycidyl 1,2-cyclohexane dicarboxylate, trimethylpropane triglycidyl ether, 1,3-bis(glycidoxypropyl)tetramethyldisiloxane, methoxymethylated urea, butoxymethylated urea, methoxymethylated thiourea, and/or the like.

The leveling agent may be used for improving coating flatness during printing and may be a commercially available suitable leveling agent.

The organic acid may include p-toluenesulfonic acid, benzenesulfonic acid, p-dodecylbenzenesulfonic acid, 1,4-naphthalenedisulfonic acid, methanesulfonic acid, a fluorinated sulfonium salt, malonic acid, citric acid, propionic acid, methacrylic acid, oxalic acid, lactic acid, glycolic acid, succinic acid, and/or a (e.g., any suitable) combination thereof, but the present disclosure is not limited thereto.

The quencher may be diphenyl (p-tolyl) amine, methyl diphenyl amine, triphenyl amine, phenylenediamine, naphthylamine, diaminonaphthalene, and/or a (e.g., any suitable) combination thereof.

An amount of these additives may be controlled or selected depending on desired or suitable properties.

In one or more embodiments, the semiconductor photoresist composition may further include a silane coupling agent as an adherence enhancer in order to improve a close-contacting force with the substrate (e.g., in order to improve adherence of the semiconductor photoresist composition to the substrate). The silane coupling agent may be for example a silane compound including a carbon-carbon unsaturated bond such as vinyltrimethoxysilane, vinyl triethoxysilane, vinyl trichlorosilane, vinyl tris(β-methoxyethoxy)silane; or 3-methacryloxypropyltrimethoxysilane, 3-acryloxypropyltrimethoxysilane, p-styryl trimethoxysilane, 3-methacryloxypropylmethyldimethoxysilane, 3-methacryloxypropylmethyl diethoxysilane; trimethoxy[3-(phenylamino)propyl]silane, and/or the like, but the present disclosure is not limited thereto.

The semiconductor photoresist composition may be formed into a pattern having a high aspect ratio without a collapse (e.g., a pattern that does not collapse). Accordingly, in order to form a fine pattern having a width of, for example, about 5 nm to about 100 nm, about 5 nm to about 80 nm, about 5 nm to about 70 nm, about 5 nm to about 50 nm, about 5 nm to about 40 nm, about 5 nm to about 30 nm, or about 5 nm to about 20 nm, the semiconductor photoresist composition may be used for a photoresist process using light in a wavelength in a range of about 5 nm to about 150 nm, for example, about 5 nm to about 100 nm, about 5 nm to about 80 nm, about 5 nm to about 50 nm, about 5 nm to about 30 nm, or about 5 nm to about 20 nm. Accordingly, the semiconductor photoresist composition according to some example embodiments may be used to realize extreme ultraviolet lithography using an EUV light source of a wavelength of about 13.5 nm.

According to some example embodiments, a method of forming patterns using the aforementioned semiconductor photoresist composition is provided. For example, the manufactured pattern may be a photoresist pattern.

The method of forming patterns according to some example embodiments includes forming an etching-objective layer on a substrate, coating the semiconductor photoresist composition on the etching-objective layer to form a photoresist layer, patterning the photoresist layer to form a photoresist pattern and etching the etching-objective layer using the photoresist pattern as an etching mask.

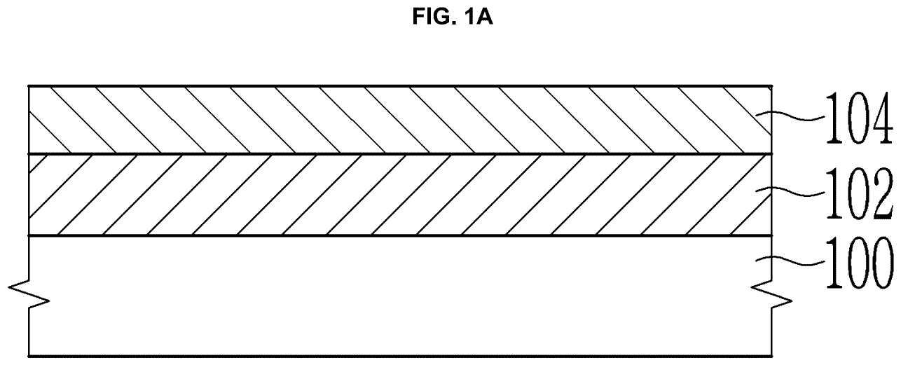

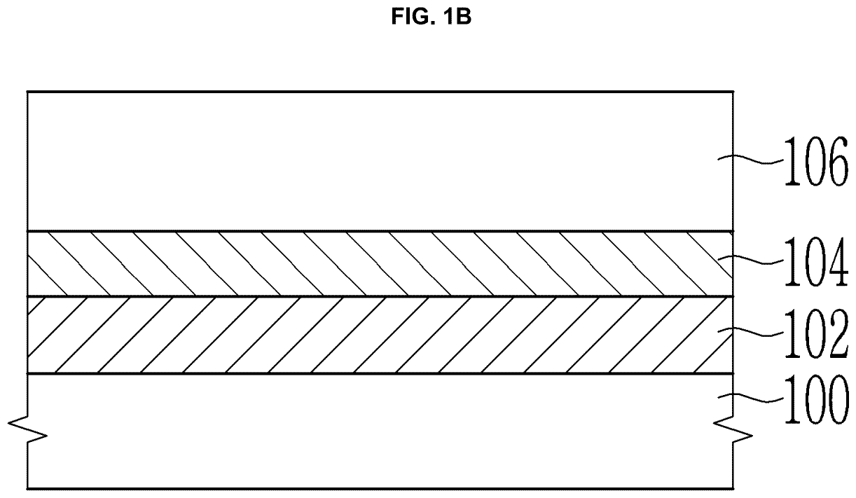

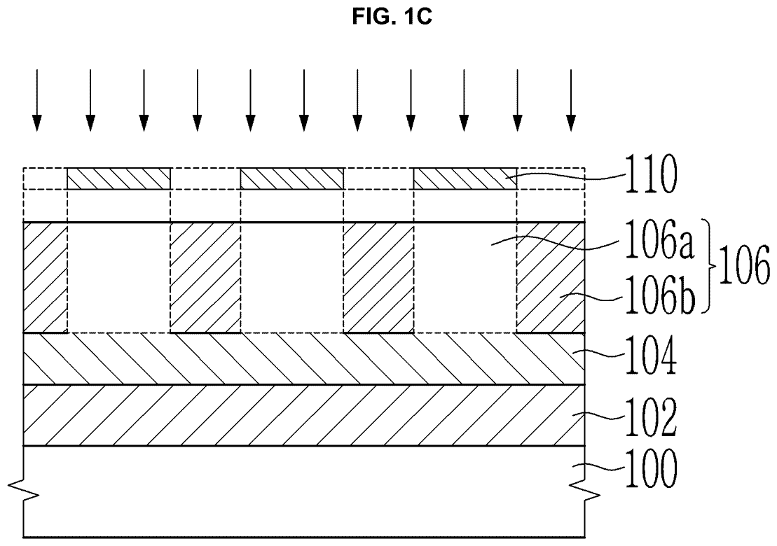

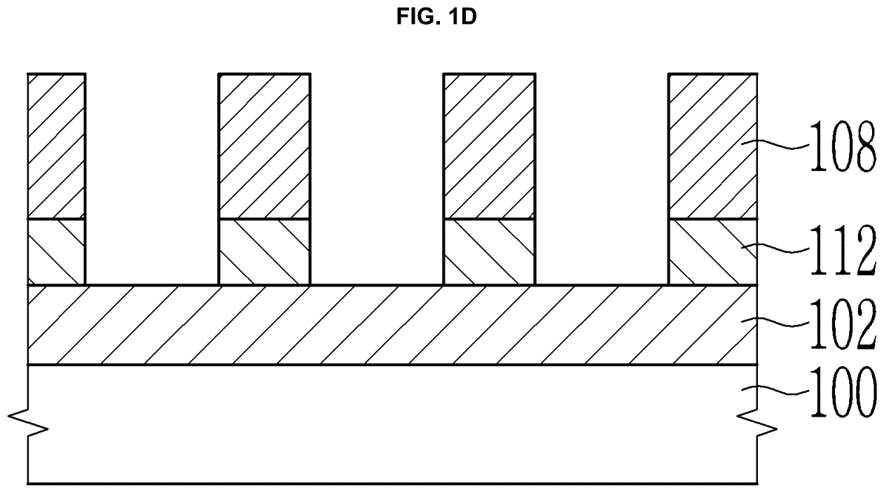

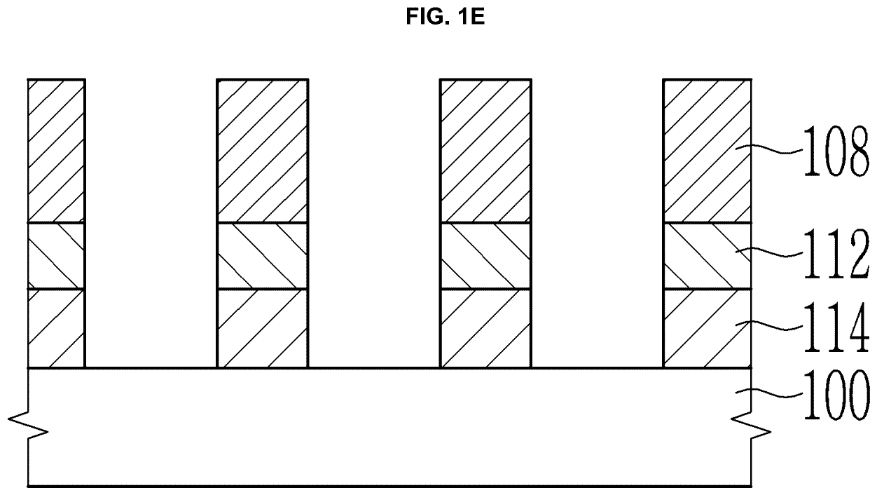

Hereinafter, a method of forming patterns using the semiconductor photoresist composition is described referring to FIGS. 1A to 1E. FIGS. 1A to 1E are cross-sectional views for explaining a method of forming patterns using a semiconductor photoresist composition according to some example embodiments.

Referring to FIG. 1A, an object for etching is prepared. The object for etching may be a thin film 102 formed on a semiconductor substrate 100. Hereinafter, as an example, the object for etching is limited to the thin film 102. A surface of the thin film 102 is washed to remove impurities and/or the like remaining thereon. The thin film 102 may be for example a silicon nitride layer, a polysilicon layer, or a silicon oxide layer.

Subsequently, the resist underlayer composition for forming a resist underlayer 104 is spin-coated on the surface of the washed thin film 102. However, one or more embodiments are not limited thereto, and one or more suitable coating methods, for example, a spray coating, a dip coating, a knife edge coating, a printing method (for example an inkjet printing and a screen printing), and/or the like may be used.

In some embodiments, the coating process of the resist underlayer may not be provided. Hereinafter, a process including the coating of the resist underlayer is described.

Then, the coated composition is dried and baked to form a resist underlayer 104 on the thin film 102. The baking may be performed at about 100° C. to about 500° C., for example, about 100° C. to about 300° C.

The resist underlayer 104 is formed between the substrate 100 and a photoresist layer 106 and thus may prevent or reduce non-uniformity and pattern formability of a photoresist line width if (e.g., when) a ray reflected from the interface between the substrate 100 and the photoresist layer 106 or a hardmask between layers is scattered into an unintended photoresist region.

Referring to FIG. 1B, the photoresist layer 106 is formed by coating the semiconductor photoresist composition on the resist underlayer 104. The photoresist layer 106 is obtained by coating the aforementioned semiconductor photoresist composition on the thin film 102 formed on the substrate 100 and then, curing it through a heat treatment.

In some embodiments, the formation of a pattern by using the semiconductor photoresist composition may include coating the semiconductor resist composition on the substrate 100 having the thin film 102 through spin coating, slit coating, inkjet printing, and/or the like and then, drying it to form the photoresist layer 106.

The semiconductor photoresist composition has already been illustrated in more detail and will not be illustrated again.

Subsequently, the substrate 100 having the photoresist layer 106 is subjected to a first baking process. The first baking process may be performed at about 80° C. to about 120° C.

Referring to FIG. 1C, the photoresist layer 106 may be selectively exposed using a patterned mask 110.

For example, the exposure may use an activation radiation with light having a high energy wavelength such as EUV (extreme ultraviolet; a wavelength of about 13.5 nm), an E-Beam (an electron beam), and/or the like as well as light having a wavelength such as an i-line (a wavelength of about 365 nm), a KrF excimer laser (a wavelength of about 248 nm), an ArF excimer laser (a wavelength of about 193 nm), and/or the like. In other words, the exposure process may utilize high-energy radiation sources like EUV (extreme ultraviolet with a wavelength of about 13.5 nm), an electron beam (E-Beam), and/or similar sources. It can also use light sources with different wavelengths, such as i-line (about 365 nm), KrF excimer laser (about 248 nm), ArF excimer laser (about 193 nm), and/or the like.

Light for the exposure according to some example embodiments may have a wavelength in a range of about 5 nm to about 150 nm or a high energy wavelength, for example, EUV (extreme ultraviolet; a wavelength of 13.5 nm), an E-Beam (an electron beam), and/or the like.

The exposed region 106b of the photoresist layer 106 has a different solubility from the unexposed region 106a of the photoresist layer 106 by forming a polymer through a crosslinking reaction such as condensation between organometallic compounds.

Subsequently, the substrate 100 is subjected to a second baking process. The second baking process may be performed at a temperature of about 90° C. to about 200° C. The exposed region 106b of the photoresist layer 106 becomes indissoluble (e.g., easily indissoluble) by a developer due to the second baking process.

In FIG. 1D, the unexposed region 106a of the photoresist layer is dissolved and removed using the developer to form a photoresist pattern 108. For example, the unexposed region 106a of the photoresist layer is dissolved and removed by using an organic solvent such as 2-heptanone and/or the like to complete the photoresist pattern 108 corresponding to the negative tone image.

A developer used in a method of forming patterns according to some example embodiments may be an organic solvent. For example, the unexposed region 106a of the photoresist layer may be dissolved and removed by using an organic solvent such as 2-heptanone and/or the like to complete the photoresist pattern 108 corresponding to the negative tone image.

However, the photoresist pattern according to some example embodiments is not necessarily limited to the negative tone image but may be formed to have a positive tone image. Herein, a developer used for forming the positive tone image may be a quaternary ammonium hydroxide composition such as tetraethylammonium hydroxide, tetrapropylammonium hydroxide, tetrabutylammonium hydroxide, and/or a (e.g., any suitable) combination thereof.

As described above, exposure to light having a high energy such as EUV (extreme ultraViolet; a wavelength of 13.5 nm), an E-Beam (an electron beam), and/or the like as well as light such as i-line (wavelength of about 365 nm), KrF excimer laser (wavelength of about 248 nm), ArF excimer laser (wavelength of about 193 nm), and/or the like may provide a photoresist pattern 108 having a width of a thickness (e.g., width) of about 5 nm to about 100 nm. For example, the photoresist pattern 108 may have a width of a thickness (e.g., width) of about 5 nm to about 90 nm, about 5 nm to about 80 nm, about 5 nm to about 70 nm, about 5 nm to about 60 nm, about 5 nm to about 50 nm, about 5 nm to about 40 nm, about 5 nm to about 30 nm, or about 5 nm to about 20 nm.

Also, the photoresist pattern 108 may have a pitch with a half-pitch of less than or equal to about 50 nm, for example less than or equal to about 40 nm, less than or equal to about 30 nm, less than or equal to about 20 nm, or less than or equal to about 15 nm, and a line width roughness of less than or equal to about 10 nm, less than or equal to about 5 nm, less than or equal to about 3 nm, or less than or equal to about 2 nm.

Subsequently, the photoresist pattern 108 is used as an etching mask to etch the resist underlayer 104. Through this etching process, an organic layer pattern 112 is formed. The organic layer pattern 112 also may have a width corresponding to that of the photoresist pattern 108.

Referring to FIG. 1E, the exposed thin film 102 is etched by applying the photoresist pattern 108 as an etching mask. As a result, the thin film is formed as a thin film pattern 114.

The etching of the thin film 102 may be for example dry etching using an etching gas and the etching gas may be for example CHF₃, CF₄, Cl₂, BCl₃ and a mixed gas thereof.

In the exposure process, the thin film pattern 114 formed by using the photoresist pattern 108 formed through the exposure process performed by using an EUV light source may have a width corresponding to that of the photoresist pattern 108. For example, the thin film pattern 114 may have a width of about 5 nm to about 100 nm which is equal to or substantially equal to that of the photoresist pattern 108. For example, the thin film pattern 114 formed by using the photoresist pattern 108 through the exposure process performed by using an EUV light source may have a width of about 5 nm to about 90 nm, about 5 nm to about 80 nm, about 5 nm to about 70 nm, about 5 nm to about 60 nm, about 5 nm to about 50 nm, about 5 nm to about 40 nm, about 5 nm to about 30 nm, or about 5 nm to about 20 nm, or less than or equal to about 20 nm, matching the width of the photoresist pattern 108.

Hereinafter, the present disclosure will be described in more detail through examples of the preparation of the aforementioned semiconductor photoresist composition. However, the present disclosure is technically not restricted by the following examples.

### Synthesis of Organometallic Compounds

### Synthesis Example 1

40.7 g of t-butylSnPh₃ and 300 g of propionic acid were added to a 250 mL two-necked round-bottom flask and heated under reflux for 24 hours.

Unreacted propionic acid was removed under reduced pressure to obtain a compound represented by Chemical Formula 6.

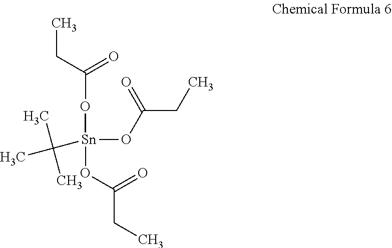 [SMILES: CCC(=O)[O][Sn]([O]C(=O)CC)([O]C(=O)CC)[C](C)(C)C]

### Synthesis Example 2

10 g of dibutyltin dichloride was dissolved in 30 mL of ether, 70 mL of a 1 M sodium hydroxide (NaOH) aqueous solution was added thereto and then, stirred for 1 hour. After the stirring, a solid produced therein was filtered, washed with 25 mL of deionized water three times, and dried at 100° C. under a reduced pressure to obtain an organometallic compound represented by Chemical Formula 7 and having a weight average molecular weight of 1,500.

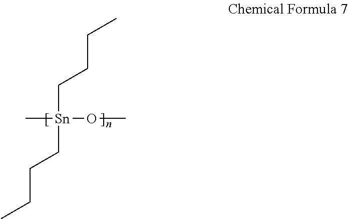

### Preparation of Semiconductor Photoresist Compositions

### Examples 1 to 9 and Comparative Examples 1 to 4

The organometallic compounds represented by Chemical Formula 6 and Chemical Formula 7 obtained in Synthesis Examples 1 and 2 were each dissolved in propylene glycol methyl ether acetate (PGMEA) at a concentration of 3%, and linear carboxylic acid compounds A1 and A2 and cyclic carboxylic acid compounds B1 to B3 were respectively added and dissolved in the weight ratios shown in Table 1, and then filtered through a 0.1 μm polytetrafluoroethylene (PTFE) syringe filter to prepare semiconductor photoresist compositions according to Examples 1 to 9 and Comparative Examples 1 to 4.

TABLE 1

| | Organometallic | Linear carboxylic | Cyclic carboxylic |
|---|---|---|---|
| | compound | acid compound | acid compound |
| | (wt %) | (wt %) | (wt %) |
| Comparative | Chemical | — | |
| Example 1 | Formula 6 | | |
| | (3.0) | | |
| Comparative | Chemical | A1 | — |
| Example 2 | Formula 6 | (0.5) | |
| | (2.5) | | |
| Comparative | Chemical | — | B1 |
| Example 3 | Formula 6 | | (0.5) |
| | (2.5) | | |
| Comparative | Chemical | A1 | B3 |
| Example 4 | Formula 6 | (0.5) | (0.2) |
| | (2.3) | | |
| Example 1 | Chemical | A1 | B1 |
| | Formula 6 | (0.5) | (0.2) |
| | (2.3) | | |
| Example 2 | Chemical | A1 | B2 |
| | Formula 6 | (0.5) | (0.2) |
| | (2.3) | | |
| Example 3 | Chemical | A2 | B1 |
| | Formula 6 | (0.5) | (0.2) |
| | (2.3) | | |
| Example 4 | Chemical | A2 | B2 |
| | Formula 6 | (0.5) | (0.2) |
| | (2.3) | | |
| Example 5 | Chemical | A1 | B2 |
| | Formula 6 | (0.35) | (0.35) |
| | (2.3) | | |
| Example 6 | Chemical | A1 | B1 |
| | Formula 6 | 0.2 | (0.5) |
| | (2.3) | | |
| Example 7 | Chemical | A1 | B2 |
| | Formula 7 | (0.5) | (0.2) |
| | (2.3) | | |
| Example 8 | Chemical | A2 | B1 |
| | Formula 7 | (0.5) | (0.2) |
| | (2.3) | | |
| Example 9 | Chemical | A2 | B2 |
| | Formula 7 | (0.5) | (0.2) |
| | (2.3) | | |

Linear carboxylic acid compound 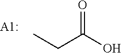 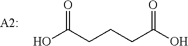
Cyclic carboxylic acid compound

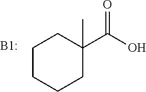

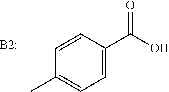

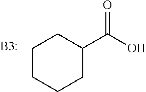

### Evaluation 1: Storage Stability Evaluation

The organometallic compounds used in Examples 1 to 9 and Comparative Examples 1 to 4 were evaluated with respect to storage stability according to the following criteria, and the results are shown in Table 2.

### Storage Stability

Each of the semiconductor photoresist compositions according to Examples 1 to 9 and Comparative Examples 1 to 4 was examined with naked eyes for a degree of precipitation if (e.g., when) allowed to stand under the condition of room temperature (20±5° C.) and then, evaluated according to the following storage criteria based on how long it took for precipitation to be observed.

### ※ Evaluation Criteria

⊚: Can be stored for more than 4 months
∘: Can be stored for 3 months or more but less than 4 months
Δ: Can be stored for less than 3 months but more than 2 weeks
X: Can be stored for less than 2 weeks

### Evaluation 2: Coating Performance Evaluation

Each of the semiconductor photoresist compositions according to Examples 1 to 9 and Comparative Examples 1 to 4 was spin-coated on a wafer at 1500 rpm for 60 seconds and baked at 110° C. for 60 seconds to form a thin film, which was imaged of with an atomic force microscopy (AFM) and/or the like, and the image was used to measure surface roughness of the thin film according to the following reference by using a software (ex. Optical Profiler), and the results are shown in Table 2.

For the surface roughness, root mean square roughness (R_{q}) refers to root mean square (rms) roughness of vertical values (e.g., heights) in the surface profile with reference to the mean line.

### Evaluation Criteria

A: Rq is less than or equal to 0.4 nm
B: Rq is greater than 0.4 nm and less than or equal to 0.6 nm
C: Rq is greater than 0.6 nm

TABLE 2

| | Storage | |
|---|---|---|
| | stability | Coatability |
| Comparative Example 1 | X | C |
| Comparative Example 2 | X | B |
| Comparative Example 3 | Δ | C |
| Comparative Example 4 | Δ | C |
| Example 1 | ⊚ | A |
| Example 2 | ⊚ | A |
| Example 3 | ⊚ | A |
| Example 4 | ⊚ | A |
| Example 5 | ⊚ | A |
| Example 6 | ⊚ | B |
| Example 7 | ⊚ | A |
| Example 8 | ⊚ | A |
| Example 9 | ⊚ | A |

From the results in Table 2, the patterns formed using the semiconductor photoresist compositions according to Examples 1 to 9 have excellent or suitable storage stability and coatability compared to Comparative Examples 1 to 4.

It will be understood that the terms “has”, “have”, “having”, “includes,” “including,” “comprises,” and/or “comprising,” when used in this specification, specify the presence of stated features, steps, operations, elements, and/or components, but do not preclude the presence or addition of one or more other features, steps, operations, elements, components, and/or groups thereof. Additionally, the terms “comprise(s)/comprising,” “include(s)/including,” or “have/has/having” include or support the terms “consisting of” and “consisting essentially of,” indicating the presence of stated features, integers, steps, operations, elements, and/or components, with or without the presence of other features, integers, steps, operations, elements, components, and/or groups thereof.

As used herein, the term “combination thereof” refers to a mixture, a laminate, a composite, a copolymer, an alloy, a blend, a reaction product, and/or the like of the constituents.

As used herein, the terms “use,” “using,” and “used” may be considered synonymous with the terms “utilize,” “utilizing,” and “utilized,” respectively. As used herein, expressions such as “at least one of”, “one of”, and “selected from”, when preceding a list of elements, modify the entire list of elements and do not modify the individual elements of the list. For example, “at least one selected from among a, b and c”, “at least one of a, b or c”, and “at least one of a, b and/or c” may indicate only a, only b, only c, both (e.g., simultaneously) a and b, both (e.g., simultaneously) a and c, both (e.g., simultaneously) b and c, all of a, b, and c, or variations thereof.

The use of “may” when describing embodiments of the inventive concept refers to “one or more embodiments of the inventive concept.”

As used herein, the term “about,” and similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Also, any numerical range recited herein is intended to include all subranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

A person of ordinary skill in the art would appreciate, in view of the present disclosure in its entirety, that each suitable feature of the various embodiments of the present disclosure may be combined or combined with each other, partially or entirely, and may be technically interlocked and operated in various suitable ways, and each embodiment may be implemented independently of each other or in conjunction with each other in any suitable manner unless otherwise stated or implied.

A device of forming patterns, or any other relevant apparatuses/devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Hereinbefore, the certain embodiments have been described and illustrated, however, it is apparent to a person with ordinary skill in the art that the present disclosure is not limited to one or more embodiments as described, and may be variously modified and transformed without departing from the spirit and scope of the present disclosure. Accordingly, the modified or transformed embodiments as such may not be understood separately from the technical ideas and aspects of the present disclosure, and the modified embodiments are within the scope of the claims of the present disclosure, and equivalents thereof.

Reference Numerals

| | |
|---|---|
| 100: substrate | 102: thin film |
| 104: resist underlayer | 106: photoresist layer |
| 106a: unexposed region | 106b: exposed region |
| 108: photoresist pattern | 112: organic layer pattern |
| 110: patterned mask | 114: thin film pattern |

What is claimed is:

1. A semiconductor photoresist composition, comprising:
an organometallic compound;
a linear carboxylic acid compound comprising at least one carboxyl group;
a cyclic carboxylic acid compound represented by Chemical Formula 1; and
a solvent: 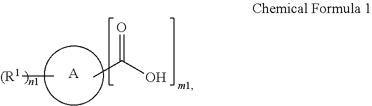
wherein, in Chemical Formula 1,
R¹ is a substituted or unsubstituted C1 to C20 alkyl group, a substituted or unsubstituted C2 to C20 alkenyl group, a substituted or unsubstituted C2 to C20 alkynyl group, a substituted or unsubstituted C3 to C20 cycloalkyl group, a substituted or unsubstituted C3 to C20 cycloalkenyl group, a substituted or unsubstituted C6 to C30 aryl group, or a substituted or unsubstituted C7 to C30 arylalkyl group,
n1 is an integer selected from among 1 to a maximum number of R¹ allowed for ring A,
ring A is a substituted or unsubstituted C3 to C20 non-aromatic carbocyclic group or a substituted or unsubstituted C6 to C30 aromatic carbocyclic group, and
m1 is an integer selected from among 1 to 3.

2. The semiconductor photoresist composition as claimed in claim 1, wherein
ring A is a substituted or unsubstituted cyclopentane group, a substituted or unsubstituted cyclohexane group, a substituted or unsubstituted heptane group, a substituted or unsubstituted octane group, a substituted or unsubstituted cyclopentene group, a substituted or unsubstituted cyclohexene group, a substituted or unsubstituted benzene group, a substituted or unsubstituted naphthalene group, a substituted or unsubstituted anthracene group, a substituted or unsubstituted phenanthrene group, a substituted or unsubstituted pyrene group, a substituted or unsubstituted triphenylene group, or a combination thereof.

3. The semiconductor photoresist composition as claimed in claim 1, wherein
the cyclic carboxylic acid compound is represented by Chemical Formula 1A or 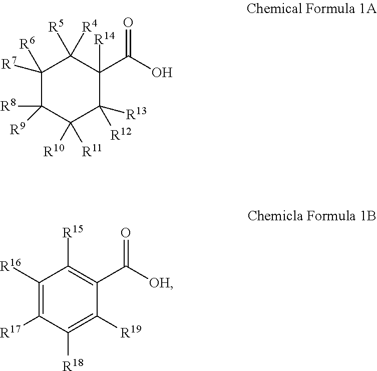
wherein, in Chemical Formula 1A and Chemical Formula 1B,
R⁴ to R¹⁹ are each independently hydrogen, a substituted or unsubstituted C1 to C20 alkyl group, a substituted or unsubstituted C2 to C20 alkenyl group, a substituted or unsubstituted C2 to C20 alkynyl group, a substituted or unsubstituted C3 to C20 cycloalkyl group, a substituted or unsubstituted C3 to C20 cycloalkenyl group, a substituted or unsubstituted C6 to C30 aryl group, or a substituted or unsubstituted C7 to C30 arylalkyl group,
at least one selected from among R⁴ to R¹⁴ is a substituted or unsubstituted C1 to C20 alkyl group, a substituted or unsubstituted C2 to C20 alkenyl group, a substituted or unsubstituted C2 to C20 alkynyl group, a substituted or unsubstituted C3 to C20 cycloalkyl group, a substituted or unsubstituted C3 to C20 cycloalkenyl group, a substituted or unsubstituted C6 to C30 aryl group, or a substituted or unsubstituted C7 to C30 arylalkyl group, and
at least one selected from among R¹⁵ to R¹⁹ is a substituted or unsubstituted C1 to C20 alkyl group, a substituted or unsubstituted C2 to C20 alkenyl group, a substituted or unsubstituted C2 to C20 alkynyl group, a substituted or unsubstituted C3 to C20 cycloalkyl group, a substituted or unsubstituted C3 to C20 cycloalkenyl group, a substituted or unsubstituted C6 to C30 aryl group, or a substituted or unsubstituted C7 to C30 arylalkyl group.

4. The semiconductor photoresist composition as claimed in claim 3, wherein
at least one selected from among R⁴ to R¹⁴ is a substituted or unsubstituted methyl group, a substituted or unsubstituted ethyl group, a substituted or unsubstituted propyl group, a substituted or unsubstituted iso-propyl group, a substituted or unsubstituted butyl group, a substituted or unsubstituted sec-butyl group, a substituted or unsubstituted iso-butyl group, a substituted or unsubstituted tert-butyl group, a substituted or unsubstituted pentyl group, or a combination thereof, and
at least one selected from among R¹⁵ to R¹⁹ is a substituted or unsubstituted methyl group, a substituted or unsubstituted ethyl group, a substituted or unsubstituted propyl group, a substituted or unsubstituted iso-propyl group, a substituted or unsubstituted butyl group, a substituted or unsubstituted sec-butyl group, a substituted or unsubstituted iso-butyl group, a substituted or unsubstituted tert-butyl group, a substituted or unsubstituted pentyl group, or a combination thereof.

5. The semiconductor photoresist composition as claimed in claim 1, wherein
the cyclic carboxylic acid compound is any one selected from among compounds listed in Group 1:

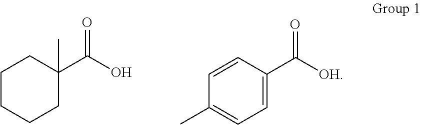

6. The semiconductor photoresist composition as claimed in claim 1, wherein
the linear carboxylic acid compound is represented by Chemical Formula 2: 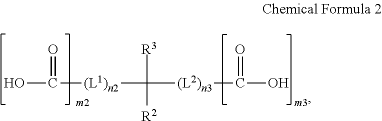
wherein, in Chemical Formula 2,
R² and R³ are each independently hydrogen, a hydroxy group, a carboxyl group, a substituted or unsubstituted C1 to C20 alkyl group, a substituted or unsubstituted C2 to C20 alkenyl group, a substituted or unsubstituted C2 to C20 alkynyl group, a substituted or unsubstituted C3 to C20 cycloalkyl group, a substituted or unsubstituted C3 to C20 cycloalkenyl group, a substituted or unsubstituted C6 to C30 aryl group, or a substituted or unsubstituted C7 to C30 arylalkyl group,
L¹ and L² are each independently a single bond, a substituted or unsubstituted C1 to C10 alkylene group, a substituted or unsubstituted C2 to C20 alkenylene group, a substituted or unsubstituted C2 to C20 alkynylene group, a substituted or unsubstituted C3 to C20 cycloalkylene group, a substituted or unsubstituted C3 to C20 cycloalkenylene group, or a substituted or unsubstituted C6 to C20 arylene group,
n2 and n3 are each independently an integer selected from among 0 to 5,
m2 and m3 are each independently 0 or 1, and
a sum of m2 and m3 is 1 or 2, and
wherein, if n2 and n3 are each an integer greater than or equal to 2, then each of L¹ and L² is the same as or different from each other.

7. The semiconductor photoresist composition as claimed in claim 6, wherein
R² and R³ are each independently hydrogen, a substituted or unsubstituted methyl group, a substituted or unsubstituted ethyl group, a substituted or unsubstituted propyl group, a substituted or unsubstituted iso-propyl group, a substituted or unsubstituted butyl group, a substituted or unsubstituted sec-butyl group, a substituted or unsubstituted iso-butyl group, a substituted or unsubstituted tert-butyl group, a substituted or unsubstituted pentyl group, or a combination thereof.

8. The semiconductor photoresist composition as claimed in claim 6, wherein
L¹ and L² are each independently a substituted or unsubstituted methylene group, a substituted or unsubstituted ethylene group, a substituted or unsubstituted propylene group, a substituted or unsubstituted butylene group, a substituted or unsubstituted pentylene group, a substituted or unsubstituted cyclopentylene group, a substituted or unsubstituted cyclopentenylene group, a substituted or unsubstituted cyclopentadienylene group, or a substituted or unsubstituted cyclohexylene group.

9. The semiconductor photoresist composition as claimed in claim 1, wherein
the linear carboxylic acid compound is any one selected from among compounds listed in Group 2:

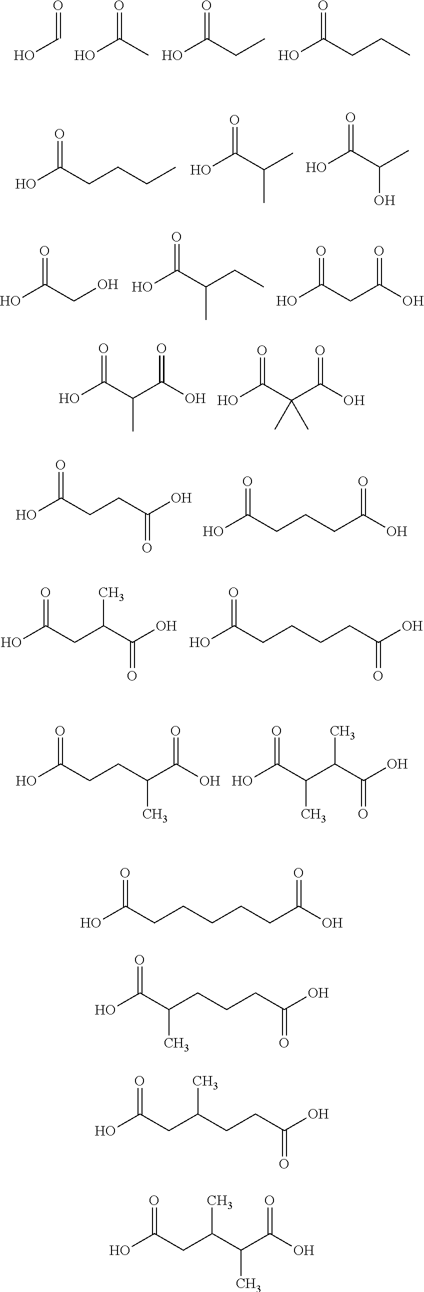

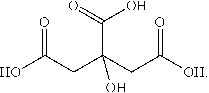 [SMILES: O=C(O)CC(O)(CC(=O)O)C(=O)O]

10. The semiconductor photoresist composition as claimed in claim 1, wherein
a weight ratio between the linear carboxylic acid compound and the cyclic carboxylic acid compound is about 9:1 to about 1:9.

11. The semiconductor photoresist composition as claimed in claim 1, wherein
the linear carboxylic acid compound and the cyclic carboxylic acid compound are about 0.01 to about 20 wt % in amount based on 100 wt % of the semiconductor photoresist composition.

12. The semiconductor photoresist composition as claimed in claim 1, wherein
the organometallic compound is about 0.5 wt % to about 30 wt % in amount based on 100 wt % of the semiconductor photoresist composition.

13. The semiconductor photoresist composition as claimed in claim 1, further comprising an additive selected from among a surfactant, a crosslinking agent, a leveling agent, an organic acid, a quencher, and a combination thereof.

14. The semiconductor photoresist composition as claimed in claim 1, wherein
the organometallic compound is an organotin compound comprising at least one of an organooxy group or an organocarbonyloxy group.

15. The semiconductor photoresist composition as claimed in claim 1, wherein
the organometallic compound is represented by Chemical Formula 3: 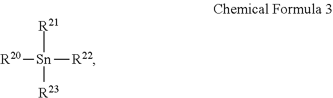
wherein, in Chemical Formula 3,
R²⁰ is selected from among a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and a substituted or unsubstituted C7 to C30 arylalkyl group,
R²¹ to R²³ are each independently:
a substituted or unsubstituted C1 to C20 alkyl group;
a substituted or unsubstituted C3 to C20 cycloalkyl group;
a substituted or unsubstituted C2 to C20 alkenyl group;
a substituted or unsubstituted C2 to C20 alkynyl group;
a substituted or unsubstituted C6 to C30 aryl group;
a substituted or unsubstituted C7 to C30 arylalkyl group;
an alkoxy or aryloxy group represented by —OR^{b}, wherein R^{b} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof;
a carboxyl group represented by —O(C═O)R^{c}, wherein R^{c} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof;
an alkylamido or dialkylamido group represented by —NR^{d}R^{e}, wherein R^{d} and R^{e} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof;
an amidato group represented by —NR^{f}(COR^{g}), wherein R^{f} and R^{g} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof;
an amidinato group represented by —NR^{h}C(NRⁱ)R^{j}, wherein R^{h}, Rⁱ and R^{j} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof;
an alkylthio or arylthiol group represented by —SR^{k}, wherein R^{k} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof; or
a thiocarboxyl group represented by —S(CO)R′, wherein R^{l} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof; and
at least one selected from among R²¹ to R²³ is selected from among the alkoxy or aryloxy group represented by —OR^{b}; the carboxyl group represented by —O(C═O)R^{c};
the alkylamido or dialkylamido group represented by —NR^{d}R^{e}; the amidato group represented by —NR^{f}(COR^{g}); the amidinato group represented by —NR^{h}C(NRⁱ)R^{j}; the alkylthio or arylthiol group represented by —SR^{k}; and the thiocarboxyl group represented by —S(CO)R^{l}.

16. The semiconductor photoresist composition as claimed in claim 15, wherein
at least one selected from among R²¹ to R²³ is selected from among the alkoxy or aryloxy group represented by —OR^{b}, and the carboxyl group represented by —O(C═O)R^{c}.

17. The semiconductor photoresist composition as claimed in claim 16, wherein
R²⁰ is a substituted or unsubstituted C1 to C8 alkyl group, a substituted or unsubstituted C3 to C8 cycloalkyl group, a substituted or unsubstituted C2 to C8 aliphatic unsaturated organic group comprising one or more double bonds or triple bonds, a substituted or unsubstituted C6 to C20 aryl group, a substituted or unsubstituted C4 to C20 heteroaryl group, a carbonyl group, an ethoxy group, a propoxy group, or a combination thereof,
R^{b} is a substituted or unsubstituted C1 to C8 alkyl group, a substituted or unsubstituted C3 to C8 cycloalkyl group, a substituted or unsubstituted C2 to C8 alkenyl group, a substituted or unsubstituted C2 to C8 alkynyl group, a substituted or unsubstituted C6 to C20 aryl group, or a combination thereof, and
R^{c} is hydrogen, a substituted or unsubstituted C1 to C8 alkyl group, a substituted or unsubstituted C3 to C8 cycloalkyl group, a substituted or unsubstituted C2 to C8 alkenyl group, a substituted or unsubstituted C2 to C8 alkynyl group, a substituted or unsubstituted C6 to C20 aryl group, or a combination thereof.

18. The semiconductor photoresist composition as claimed in claim 1, wherein
the organometallic compound is represented by Chemical Formula 4 or Chemical Formula 5: 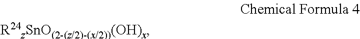
wherein, in Chemical Formula 4,
R²⁴ is a C1 to C31 hydrocarbyl group, wherein 0<z≤2 and 0<(z+x)≤4; 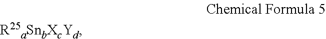
wherein, in Chemical Formula 5,
R²⁵ is a substituted or unsubstituted C1 to C20 alkyl group, a substituted or unsubstituted C3 to C20 cycloalkyl group, a substituted or unsubstituted C2 to C20 aliphatic unsaturated organic group comprising one or more double bonds or triple bonds, a substituted or unsubstituted C6 to C30 aryl group, a substituted or unsubstituted C4 to C30 heteroaryl group, a carbonyl group, an ethylene oxide group, a propylene oxide group, or a combination thereof,
X is sulfur (S), selenium (Se), or tellurium (Te),
Y is —OR^{m} or —OC(═O)Rⁿ,
R^{m} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof,
Rⁿ is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof, and
a, b, c, and d are each independently an integer selected from among 1 to 20.

19. A method comprising:
coating the semiconductor photoresist composition as claimed in claim 1 on an etching-objective layer to form a photoresist layer;
patterning the photoresist layer to form a photoresist pattern; and
etching the etching-objective layer utilizing the photoresist pattern as an etching mask,
wherein the method is a method of forming patterns.

20. The method as claimed in claim 19, further comprising forming the etching-objective layer on a substrate.
